(19) United States
(12) Patent Application Publication
(10) Pub. No.: US 2026/0021279 A1
(43) Pub. Date: Jan. 22, 2026

(54) MEDICAL DEVICE FOR USE IN CATHETER PLACEMENT

(71) Applicant: B. Braun Melsungen AG, Melsungen (DE)

(72) Inventor: Jens Wildhagen, Hannover (DE)

(21) Appl. No.: 19/271,462

(22) Filed: Jul. 16, 2025

(30) Foreign Application Priority Data
Jul. 18, 2024 (DE) 10 2024 120 392.7

(51) Int. Cl. A61M 25/06; A61M 29/02

(52) U.S. Cl. CPC A61M 25/065; A61M 29/02

(57) ABSTRACT

A medical device for use in catheter application has a longitudinal axis and a capillary portion that is elongate coaxially to the longitudinal axis between a proximal capillary end and a distal capillary end. The capillary portion has a flexurally resilient design and a capillary outside diameter. A dilator portion, which is elongate coaxially to the longitudinal axis between a proximal dilator end and a distal dilator end axially adjoining the proximal capillary end, has a dimensionally stable design and a dilator outside diameter that is larger than the capillary outside diameter at least in sections. A lumen, which is elongate coaxially to the longitudinal axis continuously between the proximal dilator end and the distal capillary end through the dilator portion and the capillary portion, is configured for receiving a cannula.

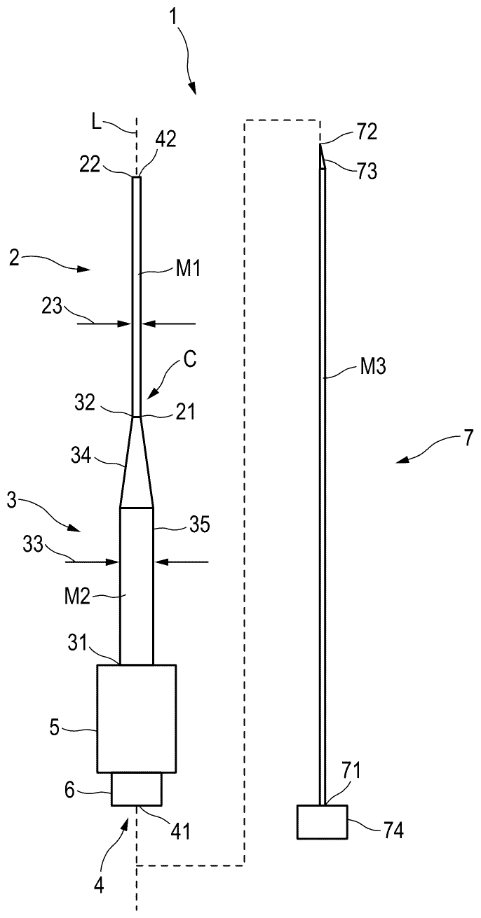

### CROSS-REFERENCE TO RELATED APPLICATION

This application claims priority under 35 U.S.C. § 119 to German Application No. 10 2024 120 392.7, filed on Jul. 18, 2024, the content of which is incorporated by reference herein in its entirety.

### FIELD

The present disclosure relates to a medical device for use in catheter application.

### BACKGROUND

It is known that a plurality of steps are required to apply a central venous catheter. In a procedure known as the Seldinger technique, a venous access is first created using a cannula. A guide wire is then inserted into the vein in question through the applied cannula. After inserting the guide wire, the cannula is pulled proximally over the guide wire and removed. Thereafter, the puncture channel of the cannula extending into the vein is widened using a dilator. For this purpose, the dilator is pushed distally over the guide wire and pressed into the puncture channel, whereby it is widened. After the puncture channel has been sufficiently widened, the dilator is pulled off proximally over the guide wire and removed. This is followed by the actual catheter application, in which the central venous catheter is pushed distally over the guide wire, inserted into the widened puncture channel and advanced along the vein to an intended position. Finally, the guide wire is pulled proximally out of the catheter and removed, and the distal catheter end is fastened to the patient's skin outside the vein.

### SUMMARY

It is an object of the present disclosure to provide a medical device that enables simplified catheter application and provides improved patient safety.

The medical device according to the present disclosure has a longitudinal axis, a capillary portion, a dilator portion and a lumen. The capillary portion is elongate coaxially to the longitudinal axis between a proximal capillary end and a distal capillary end. The capillary portion has a flexurally resilient design and a capillary outside diameter. The dilator portion is elongate coaxially to the longitudinal axis between a proximal dilator end and a distal dilator end. The distal dilator end axially adjoins the proximal capillary end. The dilator portion has a dimensionally stable design and a dilator outside diameter. The dilator outside diameter is larger than the capillary outside diameter at least in sections. The lumen is elongate coaxially to the longitudinal axis through the dilator portion and the capillary portion. The lumen is elongate continuously between the proximal dilator end and the distal capillary end. The lumen is configured to receive a cannula.

The present disclosure is based on the finding that the Seldinger technique known from the prior art requires the use of a plurality of different medical devices. These different medical devices must first be provided and used successively by the performing medical personnel during catheter application. This is time-consuming and error-prone. The present disclosure is further based on the finding that when the guide wire is inserted through the applied cannula, the vein wall opposite the puncture channel may be injured. Such injuries can be caused by unintentional movements of the applied cannula or the guide wire itself. The medical device according to the present disclosure counteracts the disadvantages described. The use of the medical device according to the present disclosure provides that the capillary portion is inserted into the vein in question through a puncture channel. The puncture channel may be produced by a cannula which, for example, is axially inserted into the lumen of the medical device and whose distal cannula tip projects beyond the distal capillary end. The cannula and the capillary portion are pushed into the body tissue together and inserted into the vein. Said cannula can then be pulled proximally out of the lumen and removed. Thereafter, the guide wire is inserted into the vein through the lumen of the medical device and thus through the dilator portion and the capillary portion. The flexurally resilient design of the capillary portion prevents injury to the vein wall in the event of unintentional movements of the medical device. In addition, the flexurally resilient capillary portion allows improved guidance of the guide wire in the longitudinal direction of the vein, which prevents injuries caused by the guide wire. After the insertion of the guide wire, the puncture channel can be widened under the influence of the dilator portion. For this purpose, the medical device is advanced distally until the dilator portion penetrates the puncture channel and widens it. Owing to the flexurally resilient design of the capillary portion, even with this distal advancement of the medical device, an injury to the vein wall is prevented. After widening the puncture channel, the medical device can be pulled off proximally over the guide wire and removed. Thereafter, the central venous catheter can be pushed over the guide wire, inserted into the widened puncture channel and advanced along the vein to an intended position. Against the background of the described mode of use of the medical device according to the present disclosure, it becomes clear that the present disclosure allows a significant increase in efficiency in catheter application. Furthermore, patient safety is improved by preventing injury to the vein wall. The capillary portion is flexurally resilient, whereas the dilator portion is dimensionally stable. In one embodiment, the capillary portion and the dilator portion are made from different materials with different moduli of elasticity, preferably from different plastics. In one embodiment, the capillary portion is softly clastic. In one embodiment, the capillary portion is formed by a tube portion, which can also be referred to in particular as a short catheter. Preferably, the capillary outside diameter is constant and/or invariable over a length of the capillary portion. Further preferably, the capillary outside diameter is round, specifically circular. In one embodiment, the dilator outside diameter is invariable and/or constant over a length of the dilator portion. In a preferred embodiment, the dilator outside diameter is variable over the length of the dilator portion. Preferably, the dilator outside diameter is round, specifically circular. The distal dilator end and the proximal capillary end directly adjoin one another axially. In other words: The proximal capillary end transitions into the distal dilator end, and vice versa. This transition is preferably radially stepless.

The medical device according to the present disclosure is particularly suitable for use in the application of a central venous catheter. However, the medical device according to the present disclosure can also be advantageously used in the application of other catheters.

In one embodiment, the dilator outside diameter varies over the length of the dilator portion and increases starting from the distal dilator end in the proximal direction. This increase is preferably continuous. Owing to the variable dilator outside diameter increasing in the distal direction, it is ensured that the dilator portion can be pressed into the puncture channel with little expenditure of force. In addition, excessive stress on the body tissue in the region of the puncture channel is counteracted.

In a further embodiment, the dilator outside diameter corresponds to the capillary outside diameter in the region of the distal dilator end, and vice versa. In this embodiment, the transition between the capillary portion and the dilator portion is radially stepless. This at any rate in practical terms. The stepless transition between the proximal capillary end and the distal dilator end prevents the distal dilator end from bluntly abutting the edge of the puncture channel when widening the puncture channel. Rather, the stepless design ensures a particularly smooth insertion of the distal dilator end into the puncture channel.

In a further embodiment, the dilator portion has a cone region and a cylinder region, wherein the cone region is proximally elongate starting from the distal dilator end with increasing dilator outside diameter and transitions into the cylinder region, wherein the dilator outside diameter is axially invariable and/or maximum in the cylinder region. The cone region allows particularly easy and smooth penetration into the puncture channel. For this purpose, the cone region has the dilator outside diameter which increases starting from the distal dilator end. The cylinder region directly adjoining the cone region proximally serves for the actual widening of the puncture channel. For this purpose, the cylinder region can be moved back and forth axially once or several times within the puncture channel. By contrast to the cone region, the dilator outside diameter is axially invariable in the cylinder region.

In a further embodiment, a length of the capillary portion is between 80% and 120%, preferably between 90% and 110%, particularly preferably between 95% and 105%, of a length of the dilator portion. In other words: The capillary portion and the dilator portion are approximately the same length. In this case, the above value ranges for the ratio between the length of the capillary portion and the length of the dilator portion have proven to be particularly advantageous.

In a further embodiment, the dilator outside diameter is at least 120%, preferably at least 150%, particularly preferably at least 200%, of the capillary outside diameter. This ensures that the puncture channel can be sufficiently widened.

In a further embodiment, the proximal capillary end and the distal dilator end are connected by means of a joining connection. In one embodiment, the joining connection is an integrally bonded connection, for example an adhesive connection or a welded connection. Alternatively or additionally, the capillary portion and the dilator portion are axially plugged together.

In a further embodiment, the medical device further comprises a handle portion. The handle portion is connected to the proximal dilator end and is configured for manual gripping by a user. The handle portion facilitates the use of the medical device, especially when widening the puncture channel. Preferably, the handle portion has an outside diameter which is larger than the (maximum) dilator outside diameter.

In a further embodiment, the medical device also has a fluid connector. The fluid connector opens distally into the lumen and is at least indirectly connected to the proximal dilator end. If the medical device has a handle portion according to the preceding embodiment, the fluid connector is preferably arranged at a proximal end of the handle portion. The fluid connector is configured for connecting to a fluid-guiding component, for example a syringe or tube. In one embodiment, the fluid connector is a Luer connector, for example a Luer lock connector or a Luer slip connector. Other types of connectors are also conceivable and possible.

In a further embodiment, the medical device also comprises a cannula, which is axially inserted or insertable into the lumen and whose distal cannula tip projects beyond the distal capillary end in the inserted state. To form the puncture channel, the distal cannula tip can be pushed into the patient's body tissue. The capillary portion is supported radially on the cannula and is inserted together with it into the body tissue and thus the vein. After forming the puncture channel, the cannula can be distally pulled out of the lumen and removed, leaving the capillary portion in the vein.

### BRIEF DESCRIPTION OF THE DRAWING

Further advantages and features of the present disclosure will become apparent from the following description and drawing.

The Figure is a schematic side view showing one embodiment of a medical device according to the present disclosure.

### DETAILED DESCRIPTION

According to the Figure, a medical device 1 for use in catheter application is provided. The medical device 1 enables efficient and particularly safe application of the catheter in question and is specifically suitable for central venous catheters.

The medical device 1 has a longitudinal axis L, a capillary portion 2, a dilator portion 3 and a lumen 4.

In the embodiment shown, the medical device 1 also has a handle portion 5, a fluid connector 6 and a cannula 7. The handle portion 5, the fluid connector 6 and the cannula 7 are each optional and therefore not present in all embodiments.

The capillary portion 2 is elongate coaxially to the longitudinal axis L. The capillary portion 2 has a proximal capillary end 21 and a distal capillary end 22. The capillary portion 2 is elongate between the proximal capillary end 21 and the distal capillary end 22. The capillary portion 2 has a flexurally resilient design. The capillary portion 2 has a capillary outside diameter 23.

The dilator portion 3 is elongate coaxially to the longitudinal axis L and thus also coaxially to the capillary portion 2. The dilator portion 3 has a proximal dilator end 31 and a distal dilator end 32. The dilator portion 3 is elongate between the proximal dilator end 31 and the distal dilator end 32. The distal dilator end 32 and the proximal capillary end 21 adjoin one another axially. The dilator portion 3 has a dimensionally stable design. The dilator portion 3 has a dilator outside diameter 33, which is larger than the capillary outside diameter 23 at least in sections.

The lumen 4 is elongate coaxially to the longitudinal axis L and thus also coaxially to the capillary portion 2 and the dilator portion 3. The lumen 4 extends continuously between the proximal dilator end 31 and the distal capillary end 22 through the dilator portion 3 and the capillary portion 2. The lumen 4 is configured for receiving the cannula 7. The lumen 4 has a proximal opening 41 and a distal opening 42. The lumen 4 is elongate continuously between the proximal opening 41 and the distal opening 42. In the embodiment shown, the proximal opening 41 is formed on the fluid connector 6. The distal opening 42 is formed at the distal capillary end 22.

In embodiments without a fluid connector, the proximal opening may be formed at the proximal dilator end or a proximal end of the handle portion.

In embodiments without a fluid connector and without a handle portion, the proximal opening may be formed at the proximal dilator end.

The flexurally resilient design of the capillary portion 2 and the dimensionally stable design of the dilator portion 3 can be achieved via a corresponding material choice. In the embodiment shown, the capillary portion 2 is made from a first material M1 and the dilator portion 3 is made from a second material M2. In the present case, the first material M1 is an elastomeric plastic, in particular with soft elastic properties. In the present case, the second material M2 is a hard plastic. The dilator portion can also be made of metal.

In the embodiment shown, the capillary outside diameter 23 is invariable over the length of the capillary portion 2. In other words, the capillary outside diameter 23 is constant. In the present case, the capillary portion 2 has a round, specifically circular, hollow cross section.

In the embodiment shown, the dilator outside diameter 33 is variable over the length of the dilator portion 3. Here, the dilator outside diameter 33 increases in the proximal direction starting from the distal dilator end 32. In the present case, this increase does not extend for instance over the entire length of the dilator portion 3, but only in regions.

In the embodiment shown, the dilator outside diameter 33 at the distal dilator end 32 corresponds at least substantially to the capillary outside diameter 23 at the proximal capillary end 21, and vice versa. At least substantially means that said outside diameters have an identical nominal dimension, although tolerance-related deviations are possible. As a result of the mutually corresponding outside diameters 23, 33 at the distal dilator end 32 and the proximal capillary end 21, an axial transition between the proximal capillary end 21 and the distal dilator end 32 is stepless in the radial direction.

In the embodiment shown, the dilator portion has a cone region 34 and a cylinder region 35.

The cone region 34 has the distal dilator end 32. The cone region 34 is elongate proximally starting from the distal dilator end 32 with increasing dilator outside diameter 33 and transitions into the cylinder region 35.

The cylinder region 35 has the proximal dilator end 31. The cylinder portion 35 is elongate between the proximal dilator end 31 and the cone region 34. The dilator outside diameter 33 is axially invariable and maximum in the cylinder region 35. In contrast, the dilator outside diameter 33 is minimal at the distal dilator end 32.

The dilator outside diameter 33 is round, specifically circular, in accordance with the capillary outside diameter 23.

In the embodiment shown, the capillary portion 2 and the dilator portion 3 are approximately the same length. Alternatively, the length of the capillary portion can be between 80% and 120%, preferably between 90% and 110%, particularly preferably between 95% and 105%, the length of the dilator portion.

The (maximum) dilator outside diameter 33 is at least 120%, preferably at least 150%, particularly preferably at least 200%, of the capillary outside diameter 23.

In the embodiment shown, the capillary portion 2 is joined to the dilator portion 3. For this purpose, a joining connection C is provided by means of which the proximal capillary end 21 and the distal dilator end 32 are joined together. In one embodiment, the joining connection is an adhesive connection. In a further embodiment, the joining connection is a welded connection. Alternatively or additionally, the capillary portion 2 and the dilator portion 3 may be axially plugged together.

The optional handle portion 5 is connected to the proximal dilator end 31 in the embodiment shown. Said connection between the dilator portion 3 and the handle portion 5 may be a one-piece connection or a joining connection suitable for the present purpose. The handle portion 5 is configured for manual gripping by a user and allows simplified manual handling of the medical device 1.

The optional fluid connector 6 is configured for connection to a fluid-guiding component, for example to a syringe or the like. The fluid connector 6 may be designed in any manner suitable for said purpose, for example as a Luer connector. However, other types of design of the fluid connector are also conceivable and possible. In the embodiment shown, the fluid connector 6 is connected to the handle portion 5, specifically to a proximal end (without reference sign) of the handle portion 5. This connection may be one-piece or formed by means of a joining connection.

The optional cannula 7 can be inserted axially into the lumen 4. In the inserted state, a distal cannula tip 72 projects beyond the distal capillary end 22.

The cannula 7 is designed in a manner known to a person skilled in the art and can also be referred to as a hollow needle. Accordingly, the cannula 7 is elongate rectilinearly between a proximal cannula end 71 and the already mentioned distal cannula tip 72 and has a ground cannula eyelet 73 in the region of the distal cannula tip 72. The cannula 7 is made from a third material M3, which in the present case is a steel suitable for medical applications. Furthermore, in the present case, the cannula 7 has a handling portion 74 which is joined to the proximal cannula end 71. In the inserted state of the cannula 7, the handling portion 74 axially abuts the fluid connector 6. The handling portion 74 may have a (further) fluid connector or be designed as such.

The medical device 1 can be used for the application of a central venous catheter as follows.

If this has not yet happened, the cannula 7 is first inserted into the lumen 4. In the inserted state, the cannula 7 supports the capillary portion 2, which is in itself flexurally resilient, in the radial direction. In other words: The capillary portion 2 is stabilized by the inserted cannula 7 and is not flexurally resilient in the inserted state of the cannula 7.

The medical device 1, with the distal cannula tip 72 leading, is brought up to a vein, which is to be punctured, of the patient concerned. The vein is punctured with the distal cannula tip 72 by advancing the medical device 1 distally. During insertion or puncturing, the cannula 7, together with the capillary portion 2 supported on it, forms a puncture channel in the patient's body tissue. This puncture channel extends into said vein. After the venous access is established in this manner, the cannula 7 can be pulled out of the lumen 4 in the proximal direction and removed. The medical device 1 can then (without the removed cannula 7) be further distally advanced, wherein the capillary portion 2 is inserted further into the vein, for example until the distal dilator end 32 bears on the puncture channel.

Thereafter, a guide wire is advanced in the distal direction through the lumen 4 and thus through the dilator portion 3 and the capillary portion 2 into the vein in question.

Thereafter or alternatively even before the insertion of the guide wire, the puncture channel is widened under the action of the dilator portion 3. This widening serves for simplified introduction of the actual central venous catheter. For widening the puncture channel, the medical device 1 is further advanced distally through the puncture channel, wherein the dilator outside diameter 33, which is larger in comparison to the capillary outside diameter 23, presses the puncture channel radially outwards and widens it in this manner.

After widening the puncture channel, the medical device 1 is pulled off the guide wire in the proximal direction and removed. Thereafter, the central venous catheter is pushed in the proximal direction over the guide wire, inserted through the widened puncture channel into the vein and advanced along the vein to its intended position.

1. A medical device for use in catheter application, the medical device comprising:
a longitudinal axis;
a capillary portion that is elongate coaxially to the longitudinal axis between a proximal capillary end and a distal capillary end, the capillary portion having a flexurally resilient design and a capillary outside diameter;
a dilator portion that is elongate coaxially to the longitudinal axis between a proximal dilator end and a distal dilator end axially adjoining the proximal capillary end, the dilator portion having a length, a dimensionally stable design, and a dilator outside diameter that is larger than the capillary outside diameter at least in sections; and
a lumen that is elongate coaxially to the longitudinal axis continuously between the proximal dilator end and the distal capillary end through the dilator portion and the capillary portion, the lumen configured for receiving a cannula.

2. The medical device according to claim 1, wherein the dilator outside diameter varies over the length of the dilator portion and increases starting from the distal dilator end in a proximal direction.

3. The medical device according to claim 1, wherein the dilator outside diameter corresponds to the capillary outside diameter in a region of the distal dilator end, and vice versa.

4. The medical device according to claim 1, wherein the dilator portion has a cone region and a cylinder region, wherein, starting from the distal dilator end, the cone region is elongate proximally with increasing dilator outside diameter and transitions into the cylinder region, and wherein the dilator outside diameter in the cylinder region is axially invariable and/or maximum.

5. The medical device according to claim 1, wherein a length of the capillary portion is between 80% and 120% of the length of the dilator portion.

6. The medical device according to claim 1, wherein the dilator outside diameter is at least 120% of the capillary outside diameter.

7. The medical device according to claim 1, wherein the proximal capillary end and the distal dilator end are connected by a joining connection.

8. The medical device according to claim 1, wherein the capillary portion and the dilator portion are axially plugged together.

9. The medical device according to claim 1, wherein the proximal capillary end and the distal dilator end are connected by a joining connection and the capillary portion and the dilator portion are axially plugged together

10. The medical device according to claim 1, further comprising a handle portion connected to the proximal dilator end and designed for manual gripping by a user.

11. The medical device according to claim 1, further comprising a fluid connector that opens proximally into the lumen, is at least indirectly connected to the dilator portion, and is configured for connection to a fluid-guiding component.

12. The medical device according to claim 1, further comprising the cannula, wherein:
the cannula is axially inserted or insertable into the lumen, and
the cannula has a distal cannula tip that projects beyond the distal capillary end in an inserted state.
